(19) United States
(12) Patent Application Publication
(10) Pub. No.: US 2005/0226878 A1
(43) Pub. Date: Oct. 13, 2005

(54) Therapeutic combinations and methods including IRM compounds

(75) Inventors: Mark A. Tomai, Woodbury, MN (US); Gary W. Gullikson, Stillwater, MN (US); David M. Hammerbeck, Houlton, WI (US); Elaine A. Egging, Woodbury, MN (US); Michael J. Reiter, New Richmond, WI (US); Christopher D. Gram, River Falls, WI (US); John P. Vasilakos, Woodbury, MN (US); Sefik S. Alkan, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/142,045

(22) Filed: Jun. 1, 2005

Related U.S. Application Data
(63) Continuation-in-part of application No. 11/001,979, filed on Dec. 2, 2004.
(60) Provisional application No. 60/526,240, filed on Dec. 2, 2003.

(51) Int. Cl. A61K 39/395; A61K 31/573; A61K 31/4745

(52) U.S. Cl. 424/145.1; 514/171; 514/291; 514/292

(57) ABSTRACT

The present invention provides therapeutic combinations that include an immune response modifier (IRM) component and an anti-inflammatory component. The inventions further provide methods of treating a condition by administering to one having the condition a therapeutic combination that includes an IRM component and an anti-inflammatory component.

### CROSS-REFERENCE TO RELATED APPLICATION

This application is a continuation-in-part of co-pending U.S. Ser. No. 11/001,979, filed Dec. 2, 2004, which claims priority to U.S. Provisional Patent Application Ser. No. 60/526,240, filed Dec. 2, 2003.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through basic immune system mechanisms known as Toll-like receptors (TLRs) to induce selected cytokine biosynthesis. They may be useful for treating a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and T_{H}2-mediated diseases (e.g., asthma, allergic rhinitis, atopic dermatitis, multiple sclerosis), and are also useful as vaccine adjuvants.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929) and more are still being discovered. Other IRMs have higher molecular weights, such as oligonucleotides, including CpGs (see, e.g., U.S. Pat. No. 6,194,388).

In view of the great therapeutic potential for IRMs, and despite the important work that has already been done, there is a substantial ongoing need to expand their uses and therapeutic benefits.

### SUMMARY

IRM compounds and anti-inflammatory compounds each can be administered to obtain certain therapeutic benefits. It has been found that therapeutic combinations of an IRM compound and an anti-inflammatory compound can provide the therapeutic benefits of treatments that include administering these compounds, but with a reduction in the side effects associated with such treatments.

The therapeutic combinations may provide effective treatment of conditions treatable with an IRM compound while ameliorating side effects associated with IRM therapies. The therapeutic combinations also may provide effective treatment for conditions treatable by administering an anti-inflammatory compound while ameliorating side effects (e.g., immunosuppression) associated with anti-inflammatory therapies.

Accordingly, the present invention provides a therapeutic combination that includes an anti-inflammatory component and an IRM component. The anti-inflammatory component can include one or more of a glucocorticoid, a non-steroidal anti-inflammatory drug, an immunosuppressant, an immunotherapeutic, or any combination thereof. In some embodiments, the IRM component includes a TLR8-selective agonist. In other embodiments, the IRM component includes an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In another aspect, the invention provides a method of treating a condition treatable with an IRM compound. Generally, the method includes administering to a subject having the condition a therapeutic combination that includes an IRM compound in an amount effective to treat the condition; and an anti-inflammatory compound in an amount effective to limit inflammation associated with treating the condition with an IRM compound. In some embodiments, the IRM component includes a TLR8-selective agonist. In other embodiments, the IRM component includes an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In another aspect, the invention provides a method of treating a condition treatable with an anti-inflammatory compound. Generally, the method includes administering to a subject having the condition a therapeutic combination that includes an anti-inflammatory compound in an amount effective to treat the condition; and an MIM compound in an amount effective to limit a side effect associated with treating the condition with the anti-inflammatory compound. In some embodiments, the IRM component includes a TLR8-selective agonist. In other embodiments, the IRM component includes an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 shows the amount of IFN-α production induced by PBMCs when stimulated with various compounds with (FIG. 1B) or without (FIG. 1A) an anti-TNF antibody.

FIG. 2 shows the amount of TNF production induced by PBMCs when stimulated with various compounds with (FIG. 2B) or without (FIG. 2A) an anti-TNF antibody.

FIG. 3 shows the amount of IFN-γ production induced by PBMCs when stimulated with various compounds with (FIG. 3B) or without (FIG. 3A) an anti-TNF antibody.

### DETAILED DESCRIPTION OF ILLUSTRATIVE EMBODIMENTS OF THE INVENTION

Therapies that include administering one or more drugs can result in side effects associated with the therapeutic drug. Side effects can vary in severity and result in, for example, patient discomfort, reduced patient compliance with the therapy, interrupting the therapy, halting the therapy, or even death.

Reducing side effects associated with a therapy can, for example, reduce the likelihood and/or frequency that the therapy must be temporarily or permanently halted due to adverse side effects. Reducing side effects associated with a therapy can, therefore, increase patient compliance, thereby reducing economic costs associated with uncompleted therapy. Reducing side effects associated with a therapy also can, for example, improve the efficacy of a therapy by either promoting completion of the therapy or allowing the therapy to include a greater therapeutic dose for a given tolerable level of therapy side effect. Consequently, methods of limiting the extent to which side effects of a drug therapy negatively influence the outcome of the therapy have substantial medical and economic benefits.

IRM compounds can possess immunostimulating activity including but not limited to antiviral and antitumor activity. Consequently, IRM compounds may be employed in drug therapies designed to treat, for example, viral infections (e.g., genital and perianal warts or Type II Herpes Simplex Virus) and neoplastic tumors (e.g., basal cell carcinoma, actinic keratosis, or melanoma). Side effects associated with certain therapies that include administering certain IRM compounds include signs of inflammation including edema, itching, and pain. While the presence of such signs are, in one respect, an indication that the drug is working as intended (i.e., by stimulating a subject's immune system to clear, for example, a viral infection), individual reactions can vary in severity. Some may desire to decrease such side effects if it is possible to do so and still maintain an effective therapy.

Abnormal regulation of inflammation can give rise to certain inflammatory disorders such as, for example, allergy, asthma, arthritis, including osteoarthritis and rheumatoid arthritis, and autoimmune conditions (e.g., lupus erythematosus). Anti-inflammatory compounds can suppress the immune system, thereby reducing an inflammatory response. Consequently, anti-inflammatory compounds may be used for therapies designed to treat inflammatory disorders by reducing the undesirably high immune activity associated with the inflammatory disorder. While a suppressed immune system can provide relief from the symptoms of an inflammatory disorder, it can also leave one more susceptible to secondary infection or neoplastic diseases, particularly when the anti-inflammatory compound is administered systemically or for a prolonged period. For example, patients taking certain anti-inflammatory compounds (TNF-α inhibitors) are at risk for certain opportunistic infections by, for example, Mycobacterium tuberculosis, Listeria monocytogenes, Pneumocystis carinii, Aspergillis fumigatus., Candida albicans., Cryptococcus neoformans, Histoplasma capsulatum, Salmonella spp., or Coccidioides immitis. Patients taking TNF-α inhibitors also may be at increased risk for developing certain other conditions including but not limited to pancytopenia and lymphoma.

The present invention provides therapeutic combinations that generally include an IRM compound and an anti-inflammatory compound. Generally speaking, the combination provides a therapeutic effect of one compound—i.e., the compound is provided in an amount effective to treat a condition (a “primary therapy” for treating a primary condition)—and the second compound is provided in an amount effective to ameliorate a side effect of administering the first compound (the “secondary therapy”). In some cases, such as, e.g., opportunistic infections, the secondary therapy may be said to be treating a secondary condition. The secondary nature of the secondary condition refers to the development of the secondary condition as a result of a primary—or prior-occurring—event such as, for example, a primary therapy, and is not intended to reflect the relative severities of the primary and secondary conditions. Therapeutic combinations of the present invention also include combinations that include two or more IRM compounds or two or more anti-inflammatory compounds.

A therapeutic combination may be provided in a single pharmaceutical composition so that both the IRM compound and the anti-inflammatory compound can be administered together. In alternative embodiments, a therapeutic combination may be provided using more than one pharmaceutical composition. In such embodiments, an IRM compound may be provided in one pharmaceutical composition and an anti-inflammatory compound may be provided in a second pharmaceutical composition so that the two compounds can be administered separately such as, for example, at different times, by different routes of administration, and the like. Thus, it also may be possible to provide the IRM compound and the anti-inflammatory compound in different dosing regimens.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

As noted above, certain IRM compounds possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1.

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; U.S. Patent Publication Nos. 2004/0091491; 2004/0147543; and 2004/0176367; and International Publication Nos. WO 2005/18551, WO 2005/18556, and WO 2005/20999.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

In some embodiments of the present invention, the IRM compound may include a 2-aminopyridine fused to a five membered nitrogen-containing heterocyclic ring, or a 4-aminopyrimidine fused to a five membered nitrogen-containing heterocyclic ring.

IRM compounds suitable for use in the invention include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

Thus, in certain embodiments, the IRM compound may be an imidazoquinoline amine such as, for example, 1-(2-methylpropyl)-1H-imidazo[4,5-c]quinolin-4-amine, 4-amino-α,α-dimethyl-2-ethoxymethyl-1H-imidazo[4,5-c]quinolin-1-ethanol, 4-amino-α,α-dimethyl-1H-imidazo[4,5-c]quinoline-1-ethanol, or 4-amino-α,α,2-trimethyl-1H-imidazo[4,5-c]quinoline-1-ethanol.

In other embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a hydroxylamine substituted imidazoquinoline amine, an oxime substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

In certain embodiments, the IRM compound may be sulfonamide substituted imidazoquinoline amine such as, for example, N-[4-(4-amino-2-ethyl-1H-imidazo[4,5-c]quinolin-1-yl)butyl]methanesulfonamide or N-{2-[4-amino-2-(ethoxymethyl)-1H-imidazo[4,5-c]quinolin-1-yl]-1,1-dimethylethyl}methanesulfonamide.

In certain alternative embodiments, the IRM compound may be a tetrahydroimidazoquinoline amine such as, for example, 4-amino-2-(ethoxymethyl)-α,α-dimethyl-6,7,8,9-tetrahydro-1H-imidazo[4,5-c]quinoline-1-ethanol.

In other alternative embodiments, the IRM compound may be an imidazonaphthyridine amine such as, for example, 2-methyl-1-(2-methylpropyl)-1H-imidazo[4,5-c][1,5]naphthyridin-4-amine or N-[4-(4-amino-2-butyl-1H-imidazo[4,5-c][1,5]naphthyridin-1-yl)butyl]-N′-cyclohexylurea.

In still other alternative embodiments, the IRM compound may be a urea substituted tetrahydroimidazoquinoline amine such as, for example, N-[4-(4-amino-2-methyl-6,7,8,9,-tetrahydro-1H-imidazo[4,5-c]quinolin-1-yl)butyl]morpholine-4-carboxamide.

Suitable IRM compounds also may include the purine derivatives, imidazoquinoline amide derivatives, benzimidazole derivatives, adenine derivatives, and oligonucleotide sequences described above.

In some embodiments of the present invention, the IRM compound may be a small molecule immune response modifier (e.g., molecular weight of less than about 1000 Daltons).

In some embodiments of the present invention, the IRM compound may be an agonist of at least one TLR such as, for example, an agonist of TLR6, TLR7, or TLR8. The IRM may also in some cases be an agonist of TLR 9. In certain embodiments, the IRM compound includes a TLR8-selective agonist. In other embodiments, the IRM compound is a TLR7-selective agonist. In still other embodiments, the IRM compound is a TLR7/8 agonist.

As used with respect to the present invention, an agonist of a TLR refers to a compound that, when combined with the TLR, can produce a TLR-mediated cellular response. A compound may be considered an agonist of a TLR regardless of whether the compound can produce a TLR-mediated cellular response by (a) directly binding to the TLR, or (b) combining with the TLR indirectly by, for example, forming a complex with another molecule that directly binds to the TLR, or otherwise resulting in the modification of another compound so that the other compound can directly bind to the TLR. A compound may be identified as an agonist of one or more particular TLRs (e.g., a TLR7 agonist, a TLR8 agonist, or a TLR7/8 agonist).

As used herein, the term “TLR8-selective agonist” refers to any compound that acts as an agonist of TLR8, but does not act as an agonist of TLR7. A “TLR7-selective agonist” refers to a compound that acts as an agonist of TLR7, but does not act as an agonist of TLR8. A “TLR7/8 agonist” refers to a compound that acts as an agonist of both TLR7 and TLR8.

A TLR8-selective agonist or a TLR7-selective agonist may act as an agonist for the indicated TLR and one or more of TLR1, TLR2, TLR3, TLR4, TLR5, TLR6, TLR9, or TLR10. Accordingly, while “TLR8-selective agonist” may refer to a compound that acts as an agonist for TLR8 and for no other TLR, it may alternatively refer to a compound that acts as an agonist of TLR8 and, for example, TLR6. Similarly, “TLR7-selective agonist” may refer to a compound that acts as an agonist for TLR7 and for no other TLR, but it may alternatively refer to a compound that acts as an agonist of TLR7 and, for example, TLR6.

The TLR agonism for a particular compound may be assessed in any suitable manner. For example, assays for detecting TLR agonism of test compounds are described, for example, in U.S. Patent Publication No. US2004/0132079, and recombinant cell lines suitable for use in such assays are described, for example, in U.S. Patent Publication No. US2004/0197865.

Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

Suitable anti-inflammatory compounds include compounds that possess anti-inflammatory activity such as, for example, glucocorticoids, non-steroidal anti-inflammatory drugs (NSAIDs), inhibitors of type 4 cyclic nucleotide phosphodiesterase (PDE4 inhibitors), beta agonists, immunosuppressants, and immunotherapeutics (i.e., antibodies). In some embodiments, the anti-inflammatory compound can act as an inhibitor of one or more pro-inflammatory biological mediators (e.g., a cytokine or enzyme) such as, for example, TNF-α, IL-1, IL-2, IL-6, IL-8, IL-12, an IL-17 family member (e.g., IL-17, IL-17F, IL-75, IL-72, IL-71, and IL-25), IL-23, MIP1-α, MCP-1, COX-2, or NFκB.

Suitable glucocorticoids include, for example, alclometasone, amcidonide, beclomethasone, betamethasone, budesonide, ciclesonide, clobetasol, clobetasone, corticosterone, cortisone, deflazacort, desonide, desoximetasone, dexamethasone, diflucotolone, diflorasone, flumethasone, flunisolide, fluocinolone, fluocinonide, fluocortolone, fluorometholone, flurandrenolone, flurandrenolide, fluticasone, halcinonide, halobetasol, hydrocortisone, methylprednisolone, mometasone, paramethasone, prednisolone, and triamcinolone.

Suitable NSAIDs include, for example, aceclofenac, acemetacin, aminopyrine, azapropazone, benzydamine, bromfenac, bufexamac, carprofen, cinnoxicam, dexketoprofen, diclofenac, diflunisal, dipyrone, etodolac, felbinac, fenbufen, fenoprofen, fentiazac, flufenamic acid, flurbiprofen, ibuprofen, indobufen, indomethacin, indoprofen, ketoprofen, meclofenamate, mefenamic acid, meloxicam, nabumetone, naproxen, niflumic acid, nimesulide, oxaprozin, oxyphenbutazone, phenylbutazone, piroxicam, salicylates (e.g., acetylated salicylates—aspirin—and nonacetylated salicylates), sulindac, suprofen, tenoxicam, tiaprofenic acid, tolfenamic acid, and tolmetin.

Suitable PDE4 inhibitors include, for example, cilomilast, roflumilast, rolipram (including R-rolipram and S-rolipram), zardaverine, CDP840, CT1731 (CDP840 enantiomer), IPL455,903 (HT-0712), RP73401, RS14203, SB207499, and T-440.

Suitable beta agonist include, for example, albuterol, bitolterol, dobutamine, isoetharine, isoproterenol, metaproterenol, pirbuterol, salmeterol, salbutamol, and terbutaline.

Suitable immunosuppressants include, for example, acetretin, alefacept, anakinra, analgesics (e.g., acetaminophen), auranofin, azathioprine, cyclophosphamide, cyclosporin, etanercept, fexofenadine, isotretinoin, leflunomide, methotrexate, minocycline, montelukast, mycophenalate, penicillamine, pimecrolimus, rosiglitazone, sirolimus, sulfasalazine, tacrolimus, tazarotene, verteporfin, zafirlukast, and zileuton.

Suitable immunotherapeutics (e.g., antibodies) include but are not limited to adalimumab, efalizumab, infliximab, omalizumab, mepolizumab, and antibodies directed against any proinflammatory molecule such as, for example, anti-TNF, anti-IL-1, anti-IL-8, anti-IL-12, anti-IL-17 family, or anti-IL-23 antibodies. As used herein, an “anti-IL-17 family antibody” may refer to an antibody directed against any IL-17 family member such as, for example, anti-IL-17, anti-IL-17F, anti-IL-75, anti-IL-72, anti-IL-71, or anti-IL-25

In one aspect, the invention provides a therapeutic combination that includes an IRM compound and an anti-inflammatory compound. In certain embodiments, the anti-inflammatory compound can include a glucocorticoid, an immunosuppressant, or an immunotherapeutic, or some combination thereof. In one embodiment, the therapeutic combination includes an IRM compound and a glucocorticoid. In an alternative embodiment, the therapeutic combination includes an immunosuppressant. In yet another alternative, the therapeutic combination includes an immunotherapeutic.

Such a therapeutic combination may be useful, for example, for reducing the extent of inflammation associated with treatments that include administering an IRM compound. For example, Tables 2 and 3 show dose dependent reductions in the synthesis of the pro-inflammatory cytokine tumor necrosis factor alpha (TNF-α) by human peripheral blood mononuclear cells (PBMCs) in response to treatment of the PBMCs with various combinations of an IRM compound and dexamethasone, a glucocorticoid anti-inflammatory compound.

The therapeutic combinations shown in Tables 2 and 3 employ the glucocorticoid in varying concentrations, and the various combinations reduce the synthesis of TNF-α in a dose-dependent manner. Thus, it may be possible to tailor the therapeutic combination to achieve a desired reduction in inflammation by varying the concentration of the glucocorticoid. Also, it may be possible to achieve a desired reduction in inflammation by using one or more alternative glucocorticoids, an immunosuppressant, or an immunotherapeutic.

Each therapeutic combination shown in Table 1 includes the IRM compound 4-amino-α,α-dimethyl-2-ethoxymethyl-1H-imidazo[4,5-c]quinolin-1-ethanol, a known TLR7/8 agonist. However, other embodiments of the invention can include a different TLR7/8 agonist, a TLR7-selective agonist, a TLR8-selective agonist, a TLR9 agonist, or any combination of two or more of the foregoing.

Such a therapeutic combination also may be useful, for example, for limiting one or more side effects that may be experienced by patients undergoing therapy designed to inhibit inflammation such as, for example, anti-TNF therapy. In some cases, the therapeutic combination may improve the efficacy of the therapy by, for example, (1) allowing the patient to tolerate a higher and, presumably, more efficacious dose of the anti-inflammatory compound, (2) providing a positive therapeutic interaction between the IRM compound and the anti-inflammatory compound, or both.

In some embodiments, the therapeutic combination can be employed to limit immunosuppression that may be experienced by subjects undergoing therapy designed to inhibit inflammation such as, for example, anti-TNF therapy. As used herein, limiting immunosuppression can refer to any desired restoration of immune function in a subject receiving a therapeutic combination of the invention compared to one receiving only anti-inflammation treatment. In some embodiments, limiting immunosuppression may manifest as reducing the likelihood or extent of a secondary condition (e.g., opportunistic infection, malignancy, or pancytopenia), or the severity of symptoms associated with a secondary condition.

Therapy designed to inhibit inflammation may be employed to treat inflammatory disorders such as, for example, rheumatoid arthritis, atopic dermatitis, asthma, allergy, and the like. Such therapy can include administering an anti-inflammatory compound (e.g., a TNF inhibitor) systemically that, in addition to suppressing the abnormal immune response that causes the condition for which the therapy was initiated, can suppress the normal immune response that would otherwise protect the patient from certain infections.

Local (i.e., non-systemic) administration of an IRM compound can provide localized immunostimulation to decrease the likelihood and extent of local opportunistic infections, or reduce the severity of opportunistic infections of, for example, the skin (e.g., atypical mycobacterial infections, Staphylococcus aureus) or lungs (e.g., atypical mycobacterial infections and opportunistic mycotic infections by, for example, Aspergillis spp., Candida spp., and Coccidioides spp.).

Local administration of certain IRM compounds (e.g., TLR8 agonists) can provide a localized induction of, for example, TNF-α that can promote a localized immune response to prophylactically or therapeutically treat an opportunistic infection without generally interfering with the effectiveness of systemic anti-inflammatory (e.g., anti-TNF) therapy.

Systemic administration of certain IRM compounds can provide systemic immunostimulation to decrease the likelihood or extent of systemic opportunistic infections, or decrease the likelihood or severity of non-infectious disorders such as, for example, certain lymphomas and pancytopenia.

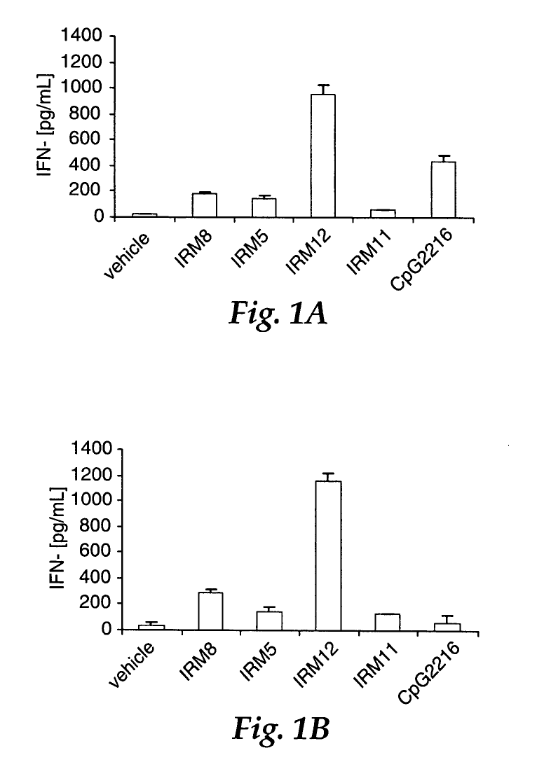

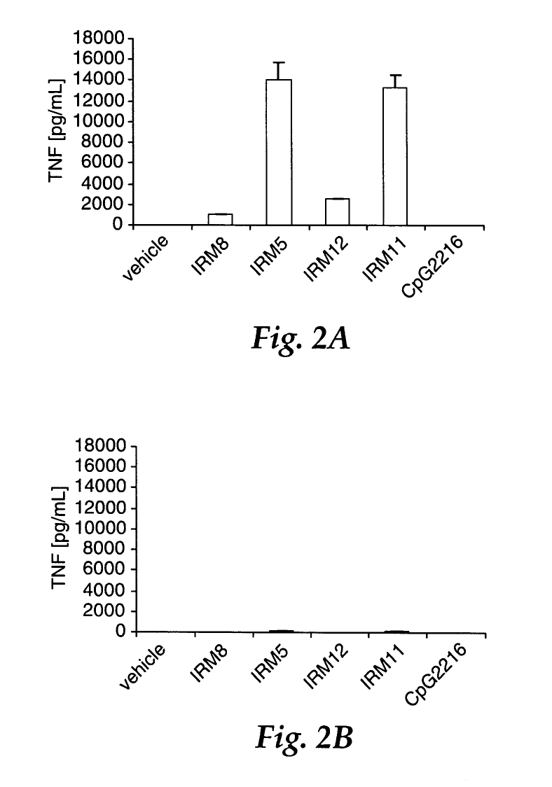

Certain IRM compounds can selectively or preferentially induce production and secretion of Type I interferons (e.g., IFN-α), while inducing little or no production and secretion of TNF-α. For example, compared to other IRM compounds, IRM8 is a relatively potent inducer of IFN-α, but a relatively poor inducer of TNF-α (See FIGS. 1A and 2A). Thus, a therapeutic combination including an anti-inflammatory compound and IRM8 (as but one example) can provide a therapeutic level of anti-TNF activity (from the anti-inflammatory compound) while inducing IFN-α-mediated immunostimulation (from IRM8), thereby reducing the immunosuppression associated with the anti-TNF therapy when it is not provided in combination with an IRM compound.

Surprisingly, in some cases the amount of IFN-α induced when the IRM compound and anti-TNF therapy are administered together is even greater than the baseline level of IFN-α production induced by the IRM compound when administered alone. Each of IRM5, IRM8, IRM11, and IRM12 exhibits increased levels of IFN-α produced when administered in combination with an anti-TNF antibody (compare FIGS. 1A and 1B).

Additionally, certain IRM compounds can induce the production and secretion of TNF-α in addition to inducing the production and secretion of Type I interferons. Surprisingly, such TNF-inducing IRM compounds still may be useful in anti-TNF therapeutic combinations because it has been found that the TNF-α production induced by such IRM compounds may be substantially limited (or even eliminated) when the IRM compound is administered in combination with an anti-TNF therapy. For example, each of IRM5, IRM11, and IRM12 induces significant levels of TNF-α when administered alone (FIG. 2A). However, when administered in combination with an anti-TNF antibody, the amount of TNF-α induced by the IRM compounds is substantially limited (FIG. 2B). Thus, such IRM compounds may ameliorate immunosuppression associated with anti-TNF therapy without substantially interfering with the anti-TNF therapy—i.e., without aggravating TNF-mediated condition.

As another example, a therapeutic combination of the invention may be used to limit the likelihood, extent, or severity of other side effects associated with therapies designed to inhibit inflammation. For example, certain IRMs may be used to limit the likelihood, frequency, and/or severity of nausea or emesis associated with such therapies.

In some embodiments, the therapeutic combination can be used to improve the efficacy of treatment for a T_{H}2-mediated inflammatory disorder. The anti-inflammatory compound may be employed to reduce a T_{H}2 immune response associated with an inflammatory disorder. The IRM compound may be provided to convert the underlying immune response away from a T_{H}2 response toward either a T_{H}1 or a TH3 immune response. Causative antigens associated with the inflammatory disorder will induce less of a T_{H}2 immune response (that which mediates the disorder) and more of a T_{H}1 or T_{H}3 immune response (those that do not mediate the disorder). The resulting improvement of the efficacy of the treatment may be in addition to, or in lieu of, the activity of limiting a side effect of the anti-inflammatory compound. Consequently, the improved efficacy of a treatment for a T_{H}2-mediated inflammatory disorder is the result of a positive therapeutic interaction between the IRM compound and the anti-inflammatory compound, and is not merely due to the patient being able to tolerate—and therefore administering to the patient—a higher dose of the anti-inflammatory compound when provided in combination with an IRM compound.

The IRM compound included in a particular therapeutic combination may vary depending upon, for example, the nature (local opportunistic infection, systemic opportunistic infection, or non-infectious) of the secondary condition sought to be controlled by the IRM compound. In some embodiments, the IRM compound may be a TLR7/8 agonist. In alternative embodiments, the IRM compound may be a TLR7-selective agonist. In additional alternative embodiments, the IRM compound may be a TLR8-selective agonist. In still other embodiments, the IRM compound may be an agonist of TLR9. A therapeutic combination of the invention may, alternatively, include two or more IRM compounds having any combination of desired TLR agonism activity (e.g., a TLR8-selective agonist and a TLR9 agonist).

The anti-inflammatory compound can be any glucocorticoid, NSAID, immunosuppressant, or immunotherapeutic anti-inflammatory compound. For embodiments in which an anti-inflammatory disorder is the primary condition to be treated using the therapeutic combination, the anti-inflammatory compound may be any anti-inflammatory suitable for treatment of the inflammatory disorder. In certain embodiments, the anti-inflammatory compound can include a glucocorticoid. In an alternative embodiment, the anti-inflammatory compound can include an immunosuppressant (e.g., etanercept). In another alternative embodiment, the anti-inflammatory compound can include an immunotherapeutic (e.g., adalirmumab, infliximab, or anti-TNF antibodies). A therapeutic combination of the invention may, alternatively, include two or more anti-inflammatory compounds.

Regardless of the particular embodiment, the therapeutic combination may be provided in a single formulation that includes both the IRM compound and the anti-inflammatory compound. Alternatively, the therapeutic combination may include a plurality of formulations. When the combination is provided in a plurality of formulations, the IRM compound and the anti-inflammatory compound may be provided in the same formulation or in different formulations. Formulations suitable for use in connection with therapeutic combinations of the invention are described in detail below.

In another embodiment, the invention includes a therapeutic combination that includes a TLR8-selective agonist and an anti-inflammatory compound. Such combinations may be useful, for example, for reducing the extent of inflammation associated with administering the TLR8-selective agonist. The anti-inflammatory compound may be any suitable anti-inflammatory compound including, for example, a glucocorticoid, an NSAID, an immunosuppressant, or an immunotherapeutic.

The therapeutic combination may be provided in a single formulation that includes both the TLR8-selective agonist and the anti-inflammatory compound. Alternatively, the therapeutic combination may include a plurality of formulations. When the combination is provided in a plurality of formulations, the TLR8-selective agonist and the anti-inflammatory compound may be provided in the same formulation or in different formulations. Formulations suitable for use in connection with therapeutic combinations of the invention are described in detail below.

The therapeutic combination may be provided in any formulation or combination of formulations suitable for administration to a subject. Suitable types of formulations are described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,186; European Patent No. EP 0 394 026; and International Patent Publication No. WO 03/045391. Each component of the combination may be provided in any suitable form including but not limited to a solution, a suspension, an emulsion, or any form of mixture. As noted above, each component of the combination may be provided together or in separate formulations. Each component of the combination may be delivered in formulation with any pharmaceutically acceptable excipient, carrier, or vehicle. For example, a formulation may be delivered in a conventional dosage form such as, for example, a cream, an ointment, an aerosol formulation, a non-aerosol spray, a gel, a lotion, a tablet, an elixir, and the like. The formulation may further include one or more additives including but not limited to adjuvants, skin penetration enhancers, colorants, flavorings, fragrances, moisturizers, thickeners, and the like.

A formulation containing one or more components of the combination may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

The amount of IRM compound and anti-inflammatory compound provided in a therapeutic combination of the invention may depend, at least in part, on whether the particular compound is being provided as a primary therapy or as a secondary therapy. Generally, the amount of compound effective to provide a primary therapy is not substantially different than the amount of compound effective to provide therapy for the primary condition outside of a therapeutic combination of the invention. In some cases, however, the amount of compound effective for treating a primary condition (i.e., for providing a primary therapy) may differ somewhat from the amount effective to treat the condition in the absence of the secondary component of the therapeutic combination.

For example, in some embodiments, the amount of compound effective to provide the primary treatment may decrease somewhat because of a positive therapeutic interaction between the primary component and the secondary component. As another example, the amount of compound effective to provide the primary treatment may increase somewhat if the secondary component acts as an antagonist of the primary component. In such cases, the secondary therapy provides sufficient benefit to offset any increase in side effects that result from increasing the amount of the primary component compound effective to provide the primary treatment.

In some embodiments, the amount of compound effective for providing the primary therapy may not necessarily change, but a higher dose may be possible because the secondary therapy provided by the secondary component of the combination permits a patient to tolerate the higher dose.

An amount of an IRM compound effective, as a secondary component of a therapeutic combination, for limiting a side effect of a primary therapy is an amount effective to reduce the likelihood, extent, or severity of the side effect (e.g., nausea, immunosuppression, etc.). For example, an amount effective for limiting nausea may be an amount effective, for example, for reducing the likelihood that a patient feels nauseous, the severity of such feelings, or reducing the frequency of, for example, vomiting. As another example, an amount effective for limiting immunosuppression may be an amount sufficient to reduce, for example, the likelihood or extent of a secondary condition, or the severity of symptoms associated with a secondary condition.

The precise amount of IRM compound for limiting a side effect in a particular therapeutic combination of the invention may vary according to factors known in the art such as, for example, the physical and chemical nature of the IRM compound; the nature of the carrier; the particular anti-inflammatory therapy with which the IRM compound is combined; the intended dosing regimen; the extent to which the subject's immune system is suppressed by the anti-inflammation therapy; the method of administering the IRM compound; whether the subject is at risk for any particular secondary condition and, if so, the identity of such a secondary condition; and the species to which the formulation is being administered. Accordingly, it is not practical to set forth generally the amount that constitutes an amount of IRM compound effective for limiting immunosuppression for all possible applications. Those of ordinary skill in the art, however, can readily determine the appropriate amount with due consideration of such factors.

In some embodiments, the methods of the present invention include administering sufficient IRM compound to provide a dose of, for example, from about 100 ng/kg to about 50 mg/kg to the subject, although in some embodiments the methods may be performed by administering IRM compound in concentrations outside this range. In some of these embodiments, the method includes administering sufficient IRM compound to provide a dose of from about 10 μg/kg to about 5 mg/kg to the subject, for example, a dose of from about 100 μg/kg to about 1 mg/kg.

The IRM compound may be administered to a subject in a formulation that includes, for example, from about 0.001% to about 10% IRM compound (unless otherwise indicated, all percentages provided herein are weight/weight with respect to the total formulation) to the subject, although in some embodiments the IRM compound may be administered using a formulation that provides IRM compound in a concentration outside of this range. In certain embodiments, the method includes administering to a subject a formulation that includes from about 0.01% to about 1% IRM compound, for example, a formulation that includes from about 0.1% to about 0.5% IRM compound.

An amount of an anti-inflammatory compound effective, as a secondary component of a therapeutic combination, for limiting a side effect of a primary therapy is an amount effective to reduce the likelihood, extent, or severity of the side effect (e.g., edema, itching, pain, etc.). For example, an amount of an anti-inflammatory compound effective for reducing, for example, inflammation associated with administering an IRM compound may be an amount sufficient to reduce the likelihood or extent of inflammation in a subject receiving a therapeutic combination of the invention compared to receiving only the IRM compound.

The precise amount of anti-inflammatory compound for limiting a side effect (e.g., reducing inflammation) in a particular therapeutic combination of the invention may vary according to factors known in the art such as, for example, the physical and chemical nature of the anti-inflammatory compound; the potency of the anti-inflammatory compound; the nature of the carrier; the particular IRM compound with which the anti-inflammatory compound is combined; the intended dosing regimen; the nature of the subject's immune system (e.g., suppressed, compromised, stimulated); the method of administering the anti-inflammatory compound; and the species to which the formulation is being administered. Accordingly, it is not practical to set forth generally the amount that constitutes an amount of anti-inflammatory compound effective for limiting immunosuppression for all possible applications. Those of ordinary skill in the art, however, can readily determine the appropriate amount with due consideration of such factors.

In some embodiments, the methods of the present invention include administering sufficient anti-inflammatory compound to provide a dose of, for example, from about 100 ng/kg to about 50 mg/kg to the subject, although in some embodiments the methods may be performed by administering anti-inflammatory compound in concentrations outside this range. In some of these embodiments, the method includes administering sufficient anti-inflammatory compound to provide a dose of from about 10 ag/kg to about 10 mg/kg to the subject. In certain embodiments, the method includes administering sufficient anti-inflammatory compound to provide a dose of from about 50 μg/kg to about 2.5 mg/kg, for example, a dose of from about 200 μg/kg to about 1 mg/kg.

The anti-inflammatory compound may be administered to a subject in a formulation that includes, for example, from about 0.001% to about 10% anti-inflammatory compound to the subject, although in some embodiments the anti-inflammatory compound may be administered using a formulation that provides the compound in a concentration outside of this range. In some embodiments, the method includes administering to a subject a formulation that includes from about 0.01% to about 2.5% anti-inflammatory compound. In certain embodiments, the method includes administering to a subject a formulation that includes from about 0.05% to about 1.0% anti-inflammatory compound, for example, a formulation that includes from about 0.1% to about 0.5% anti-inflammatory compound.

The dosing regimen of each component of a therapeutic combination of the invention may be the same as, or different than, the dosing regimen of the other component. Specifically, the dosing regimen of a component may be dependent upon whether a particular component is providing a primary therapy (i.e., the primary component) or is intended to reduce or limit a side effect associated with the primary therapy (i.e., the secondary component). In either case, the dosing regimen for a compound may depend at least in part on many factors known in the art such as, for example, the physical and chemical nature of the compound, the chemical and physical nature of the other compound of the therapeutic combination, the nature of the carrier, the amount of the other compound of the therapeutic combination being administered, the state of the subject's immune system (e.g., suppressed, compromised, stimulated), and the method of administering the compound, the presence and extent of any interactions between the compound of the primary component and the compound of the secondary component, and the species to which the formulation is being administered.

For the primary component of the therapeutic combination, additional factors include, for example, the typical dosing regimen for the compound used to treat the primary condition.

For the secondary component of the therapeutic combination, additional factors include, for example, the severity of side effect associated with the primary therapy.

Accordingly it is not practical to set forth generally the dosing regimen for each component for all possible therapeutic combinations of the invention. Those of ordinary skill in the art, however, can readily determine the appropriate dosing regimen with due consideration of such factors.

In some embodiments of the invention, the secondary component compound may be administered, for example, from once to multiple times per day. For example, the secondary component compound may be administered from about once per week to about four times per day, although in some embodiments the methods of the present invention may be performed by administering the secondary component compound at a frequency outside this range. In certain embodiments, the secondary component compound is administered from about three times per week to about twice per day. In one particular embodiment, the secondary component compound may be administered on an “as needed” basis. In an alternative embodiment, the secondary component may be administered once per day for three days per week. In an alternative embodiment, the secondary component compound may be administered once per day for four days per week. In another alternative embodiment, the secondary component compound may be administered once per day for five days per week. In another alternative embodiment, the secondary component compound may be administered once per day each day of the week. In yet another alternative embodiment, the secondary component compound may be administered twice per day at least one day per week. Whenever the secondary component compound is administered on more than one day per week and less than seven days per week, the compound may be administered on consecutive days or non-consecutive days, as desired.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The IRM compounds used in the examples are identified in Table 1.

TABLE 1
IRM Compounds

| Compound | Chemical Name | Reference |
|---|---|---|
| IRM1 | 4-amino-α,α-dimethyl-2-ethoxymethyl-1H- | U.S. Pat. No. 5,389,640 |
| | imidazo[4,5-c]quinolin-1-ethanol | Example 99 |
| IRM2 | 4-amino-α,α,2-trimethyl-1H-imidazo[4,5- | U.S. Pat. No. 5,266,575 |
| | c]quinoline-1-ethanol | Example C1 |
| IRM3 | 1-(2-methylpropyl)-1H-imidazo[4,5-c]quinolin-4- | U.S. Pat. No. 4,689,338 |
| | amine | Example 99 |
| IRM4 | 4-amino-α,α-dimethyl-1H-imidazo[4,5- | U.S. Pat. No. 4,689,338 |
| | c]quinoline-1-ethanol | Example 189 |
| IRM5 | 4-amino-2-(ethoxymethyl)-α,α-dimethyl-6,7,8,9- | U.S. Pat. No. 5,352,784 |
| | tetrahydro-1H-imidazo[4,5-c]quinoline-1-ethanol | Example 91 |
| IRM6 | 2-methyl-1-(2-methylpropyl)-1H-imidazo[4,5-c] | U.S. Pat. No. 6,194,425 |
| | [1,5]naphthyridin-4-amine | Example 36 |
| IRM7 | N-[4-(4-amino-2-butyl-1H-imidazo[4,5- | U.S. Pat. No. 6,194,425 |
| | c][1,5]naphthyridin-1-yl)butyl]-N′- | Example 48 |
| | cyclohexylurea | |
| IRM8 | N-[4-(4-amino-2-ethyl-1H-imidazo[4,5- | U.S. Pat. No. 6,677,349 |
| | c]quinolin-1-yl)butyl]methanesulfonamide | Example 236 |
| IRM9 | N-[4-(4-amino-2-methyl-6,7,8,9,-tetrahydro-1H- | U.S. Pat. No. 6,573,273 |
| | imidazo[4,5-c]quinolin-1-yl)butyl]morpholine-4- | Example 170 |
| | carboxamide | |
| IRM10 | N-{2-[4-amino-2-(ethoxymethyl)-1H- | U.S. Pat. No. 6,677,349 |
| | imidazo[4,5-c]quinolin-1-yl]-1,1- | Example 268 |
| | dimethylethyl}methanesulfonamide | |
| IRM11 | 2-propylthiazolo[4,5-c]quinolin-4-amine | U.S. Pat. No. 6,110,929 |
| | | Example 12 |
| IRM12 | N-(2-{2-[4-amino-2-(2-methoxyethyl)-1H- | U.S. Pat. No. 6,656,938 |
| | imidazo[4,5-c]quinolin-1- | Example 5 |
| | yl]ethoxy}ethyl)morpholine-4-carboxamide | |

### Example 1

Whole blood from healthy human donors was collected by venipuncture into EDTA vacutainer tubes (Becton Dickinson Labware, Lincoln Park, N.J.). Peripheral blood mononuclear cells (PBMCs) are separated from whole blood by density gradient centrifugation using HISTOPAQUE-1077 (Sigma-Aldrich Chemical Co., St. Louis, Mo.). The PBMCs are washed twice with Hank's Balanced Salts Solution (Celox Laboratories, Inc., Hopkins, Minn.) and then are suspended at 3-4×10⁶ cells/mL in RPMI complete culture medium (Celox Laboratories, Inc., Hopkins, Minn.). The PBMC suspension was added to 48 well flat bottom sterile tissue culture plates (Becton Dickinson Labware, Lincoln Park, N.J.) containing an equal volume of RPMI complete media containing dexamethasone (Sigma Chemical Co., St. Louis, Mo.) at one of the dexamethasone concentrations indicated in Table 2.

After one hour, IRMI was added to a final concentration of 1 μM, and then incubated at 37° C. for an additional 24 hours. Following incubation the cells were centrifuged for 5-10 minutes at 1000 rpm (˜200×g) at 4° C. The cell-free culture supernatant is removed with a sterile polypropylene pipette and transferred to sterile polypropylene tubes. Samples were maintained at −70° C. until analysis.

The samples were analyzed for IFN-α and TNF-α secreted into the culture medium. The concentration of secreted IFN-α was determined by ELISA using a Human Multi-Species kit (PBL Biomedical Laboratories, Piscataway, N.J.). Secreted TNF-α was assayed by ELISA (R&D Systems, Minneapolis, Minn.). Results are shown in Table 2.

TABLE 2

| Treatment | TNF (pg/mL) | IFN (U/mL) |
|---|---|---|
| Medium | 0 | 0 |
| IRM1 (1 μM) | 3000 | 959 |
| IRM1 (1 μM) + dexamethasone (100 nM) | 156 | 421 |
| IRM1 (1 μM) + dexamethasone (10 nM) | 114 | 421 |
| IRM1 (1 μM) + dexamethasone (1.0 nM) | 247 | 185 |
| IRM1 (1 μM) + dexamethasone (0.1 nM) | 1260 | 185 |
| IRM1 (1 μM) + dexamethasone (0.01 nM) | 3000 | 421 |

### Example 2

Human PBMCs were collected and prepared as described in Example 1. The cells were incubated in dexamethasone at one of the concentration indicated in Table 3. After one hour, the cells were treated with IRM compound, LPS, or left unstimulated, and then incubated for an additional 24 hours.

Secreted TNF-α was assayed by ELISA (Biosource International, Inc., Camarillo, Calif.). Results are expressed as pg/mL and are shown in Table 3.

TABLE 3

| | 0 μM | | | |
|---|---|---|---|---|
| Treatment | Dex. | 0.01 μM Dex. | 0.1 μM Dex. | 1.0 μM Dex. |
| Medium | 0 | 0 | 45 | 0 |
| IRM2 (1.0 μg/mL) | 1570 | 775 | 224 | 191 |
| LPS (0.1 μg/mL) | 3300 | 1630 | 1620 | 874 |

### Example 3

A dexamethasone solution was prepared in saline and administered orally (3 mg/kg) to male CFW mice (Charles River Laboratories, Inc., Wilmington, Mass.) once or once daily for five days. Thirty minutes after the final administration of dexamethasone, the mice were challenged with a solution of IRM2 prepared in saline to provide a dose of 10 mg/kg. The mice were bled either 2 hours or 3 hours after being challenged with IRM2. Serum samples were analyzed by for TNF by ELISA as described in Example 1. The results are expressed as pg/mL and are shown in Table 4.

TABLE 4

| | 2 hrs. post- | 3 hrs. post- |
|---|---|---|
| Treatment | challenge | challenge |
| Unchallenged | 5.3 | * |
| IRM2 | 2983 | 487 |
| IRM2 + 1 × dexamethasone | 1525 | 248 |
| IRM2 + 5 × dexamethasone | 306 | 78 |

* not analyzed

### Example 4

Human PBMCs were collected and prepared as described in Example 1. The cells were incubated with an anti-TNF monoclonal antibody (mouse anti-human TNF, Promega Corp., Madison, Wis.). After one hour, the cells were treated with IRM compound, LPS, or left unstimulated, and then incubated for an additional 24 hours.

Secreted TNF-α and IL-6 were assayed by ELISA (Biosource International, Inc., Camarillo, Calif.). Results are expressed in pg/mL for each cytokine.

Secreted IFN was assayed using a virus neutralization bioassay using A549 human lung carcinoma cells challenged with encephalomyocarditis. The details of the bioassay method have been described by G. L. Brennan and L. H. Kronenberg in “Automated Bioassay of Interferons in Micro-test Plates”, Biotechniques, June/July, 78, 1983, incorporated herein by reference. Briefly stated the method is as follows: A549 cells are incubated with dilutions of samples or a standard interferon at 37° C. for 24 hours. The incubated cells are then infected with an inoculum of encephalomyocarditis virus. The infected cells are incubated for an additional 24 hours at 37° C. before evaluating for viral cytopathic effect. The viral cytopathic effect is quantified by staining with crystal violet followed by visual scoring of the plates. Results are expressed as alpha reference units/mL based on the value obtained for NIH Human Leukocyte IFN standard.

Results are shown in Table 5.

TABLE 5

| | TNF (pg/mL) | | IL-6 (pg/mL) | | IFN (U/mL) | |
|---|---|---|---|---|---|---|
| Treatment | No Ab | +Ab | No Ab | +Ab | No Ab | +Ab |
| Medium | 5 | 0 | 0 | 702 | 0 | 0 |
| IRM2 (1.0 μg/mL) | 1340 | 61 | 20137 | 24189 | 460 | 290 |
| LPS (0.1 μg/mL) | 1060 | 61 | 22979 | 26111 | 22 | 8.8 |

### Example 5

Human PBMCs are collected and prepared as described in Example 1. After one hour, IRM3, IRM4, IRM5, IRM6, IRM7, IRM8, IRM9, or IRM10 is added to a final concentration of 1 μM, and then incubated at 37° C. for an additional 24 hours. The samples are analyzed for IFN-α and TNF-α as described in Example 1. Results will show inhibition of IRM-induced TNF-α by dexamethasone in a dose dependent manner.

### Example 6

Whole blood from healthy human donors was collected by venipuncture into EDTA vacutainer tubes (Becton Dickinson Labware, Lincoln Park, N.J.). Peripheral blood mononuclear cells (PBMCs) are separated from whole blood by density gradient centrifugation using HISTOPAQUE-1077 (Sigma-Aldrich Chemical Co., St. Louis, Mo.). The PBMCs are washed twice with Phosphate Buffered Saline (PBS, Biosource, Camarillo, Calif.) and then are suspended at 5×10⁶ cells/mL in RPMI complete culture medium (Celox Laboratories, Inc., Hopkins, Minn.). One milliliter of the PBMC suspension and 1.5 milliliter of RPMI complete media were added to each well of a 24 well flat bottom sterile tissue culture plate (Becton Dickinson Labware, Lincoln Park, N.J.) for a final PBMC concentration of 2×10⁶ cells/mL.

PBMCs were treated with 1 μg/mL of anti-TNF antibody (Cat#16-7348-81, eBioscience, San Diego, Calif.) or 1 μg/mL of an IgG1 isotype control antibody (Cat# 554721, BD Pharmigen, San Diego, Calif.) and then incubated at 37° C. for one hour. Following the 1 hour incubation, PBMCs were treated with 3 μM IRM5, 3 μM IRM8, 3 μM IRM11, 3 μM IRM12, 1 μM CpG2216 (Invitrogen, Carlsbad, Calif.) or untreated (vehicle) and then incubated at 37° C. for 18 to 24 hours. Following incubation the cells were centrifuged for 5-10 minutes at 1000 rpm (˜200×g) at 4° C. The cell-free culture supernatant was removed with a sterile polypropylene pipette and transferred to sterile polypropylene tubes. Samples were maintained at −70° C. until analysis.

The samples were analyzed for IFN-α, TNF-α and IFN-γ secreted into the culture medium. The concentration of secreted IFN-α was determined by ELISA using a Human Multi-Species kit (PBL Biomedical Laboratories, Piscataway, N.J.). Secreted TNF-α and IFN-γ were analyzed using a human-specific TNF-α BV™ or IFN-γ BV™ immunoassay (BioVeris Corp., Gaithersburg, Md.).

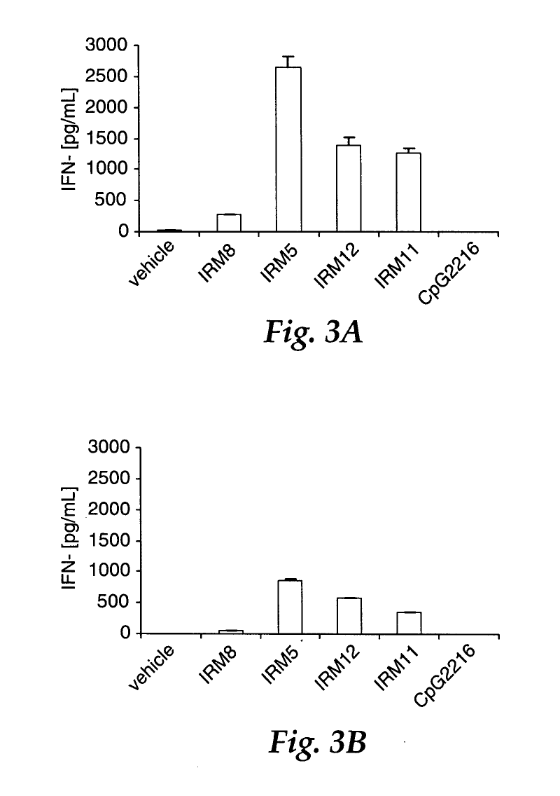

Results of IFN-α, TNF-α and IFN-γ cytokine induction by PBMCs in the presence of the isotype control antibody are found in FIGS. 1A, 2A, and 3A, respectively. Results of IFN-α, TNF-α and IFN-γ cytokine induction by PBMCs in the presence of the anti-TNF antibody are found in FIGS. 1B, 2B, and 3B, respectively.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A therapeutic combination comprising:
an IRM compound; and
an anti-inflammatory component that comprises an immunotherapeutic that comprises an anti-IL-17 family antibody or an anti-IL-23 antibody.

2. The therapeutic combination of claim 1 wherein the IRM component comprises an agonist of at least one of TLR7, TLR8, and TLR9.

3. The therapeutic combination of claim 2 wherein the IRM component comprises an agonist of at least TLR7 or TLR8.

4. The therapeutic combination of claim 3 wherein the IRM component comprises a TLR8-selective agonist.

5. The therapeutic combination of claim 3 wherein the IRM component comprises a TLR7-selective agonist.

6. The therapeutic combination of claim 3 wherein the IRM component comprises a TLR7/8 agonist.

7. The therapeutic combination of claim 1 comprising a plurality of formulations.

8. The therapeutic combination of claim 7 wherein a first formulation comprises the IRM compound and a second formulation comprises the anti-inflammatory component.

9. The therapeutic combination of claim 1 wherein the anti-inflammatory component further comprises a glucocorticoid.

10. The therapeutic combination of claim 9 wherein the glucocorticoid comprises alclometasone, amcidonide, beclomethasone, betamethasone, budesonide, ciclesonide, clobetasol, clobetasone, corticosterone, cortisone, deflazacort, desonide, desoximetasone, dexaamethasone, diflucotolone, diflorasone, flumethasone, flunisolide, fluocinolone, fluocinonide, fluocortolone, fluorometholone, flurandrenolone, flurandrenolide, fluticasone, halcinonide, halobetasol, hydrocortisone, methylprednisolone, mometasone, paramethasone, prednisolone, or triamcinolone.

11. The therapeutic combination of claim 1 wherein the anti-inflammatory component further comprises a non-steroidal anti-inflammatory drug.

12. The therapeutic combination of claim 11 wherein the non-steroidal anti-inflammatory drug comprises aceclofenac, acemetacin, aminopyrine, azapropazone, benzydamine, bromfenac, bufexamac, carprofen, cinnoxicam, dexketoprofen, diclofenac, diflunisal, dipyrone, etodolac, felbinac, fenbufen, fenoprofen, fentiazac, flufenamic acid, flurbiprofen, ibuprofen, indobufen, indomethacin, indoprofen, ketoprofen, meclofenamate, mefenamic acid, meloxicam, nabumetone, naproxen, niflumic acid, nimesulide, oxaprozin, oxyphenbutazone, phenylbutazone, piroxicam, a salicylate, sulindac, suprofen, tenoxicam, tiaprofenic acid, tolfenamic acid, or tolmetin.

13. The therapeutic combination of claim 1 wherein the anti-inflammatory component further comprises an immunosuppressant.

14. The therapeutic combination of claim 13 wherein the immunosuppressant comprises acetretin, alefacept, anakinra, an analgesic, auranofin, azathioprine, cyclophosphamide, cyclosporin, etanercept, isotretinoin, leflunomide, methotrexate, minocycline, montelukast, mycophenalate, penicillamine, pimecrolimus, rosiglitazone, sirolimus, sulfasalazine, tacrolimus, tazarotene, verteporfin, zafirlukast, or zileuton.

15. The therapeutic combination of claim 1 wherein the anti-inflammatory component further comprises a second immunotherapeutic.

16. The therapeutic combination of claim 15 wherein the immunotherapeutic comprises an antibody directed against a proinflammatory molecule.

17. The therapeutic combination of claim 16 wherein the immunotherapeutic comprises adalimumab, efalizumab, infliximab, omalizumab, or mepolizumab.

18. The therapeutic combination of claim 1 wherein the IRM compound comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, a tetrahydropyrazolonaphthyridine amine, or a 1H-imidazo dimer fused to a pyridine amine, a quinoline amine, a tetrahydroquinoline amine, a naphthyridine amine, or a tetrahydronaphthyridine amine.

19. The therapeutic combination of claim 1 wherein the IRM compound comprises an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, a tetrahydropyrazolonaphthyridine amine, or a 1H-imidazo dimer fused to a pyridine amine, a quinoline amine, a tetrahydroquinoline amine, a naphthyridine amine, or a tetrahydronaphthyridine amine.

20. A method of treating a condition treatable with an IRM compound, the method comprising administering to a subject having the condition a therapeutic combination that comprises
(a) an IRM compound in an amount effective to treat the condition; and
(b) an anti-inflammatory compound that an amount effective to limit a side effect of administering the IRM compound, wherein the anti-inflammatory compound comprises an anti-IL-17 family antibody or an anti-IL-23 antibody.

21. The method of claim 20 wherein the IRM compound and the anti-inflammatory compound are administered at different sites.

22. The method of claim 20 wherein the IRM compound and the anti-inflammatory compound are administered at different times.

23. The method of claim 20 wherein the IRM compound comprises an agonist of at least one of TLR7, TLR8, and TLR9.

24. The method of claim 23 wherein the IRM compound comprises an agonist of TLR7 or TLR8.

25. The method of claim 24 wherein the IRM compound comprises a TLR8-selective agonist.

26. The method of claim 24 wherein the IRM compound comprises a TLR7-selective agonist.

27. The method of claim 24 wherein the IRM compound comprises a TLR7/8 agonist.

28. The method of claim 20 wherein the IRM compound comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, a tetrahydropyrazolonaphthyridine amine, or a 1H-imidazo dimer fused to a pyridine amine, a quinoline amine, a tetrahydroquinoline amine, a naphthyridine amine, or a tetrahydronaphthyridine amine.

29. The method of claim 20 wherein the IRM compound comprises an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, a tetrahydropyrazolonaphthyridine amine, or a 1H-imidazo dimer fused to a pyridine amine, a quinoline amine, a tetrahydroquinoline amine, a naphthyridine amine, or a tetrahydronaphthyridine amine.

30. A method of ameliorating inflammation associated with administering an IRM compound to provide a medical treatment, the method comprising:
administering the IRM compound in an amount effective to provide the medical treatment; and
administering an anti-inflammatory compound in an amount effective to reduce the inflammation associated with administering the IRM, wherein the anti-inflammatory compound comprises an anti-IL-17 antibody or an anti-IL-23 antibody.

31. A method of treating a condition treatable with an anti-inflammatory compound, the method comprising administering to a subject having the condition a therapeutic combination that comprises
(a) an anti-inflammatory compound in an amount effective to treat the condition; and
(b) an IRM compound in an amount effective to limit immunosuppression.

32. The method of claim 31 wherein the IRM compound and the anti-inflammatory compound are administered at different sites.

33. The method of claim 31 wherein the IRM compound and the anti-inflammatory compound are administered at different times.

34. The method of claim 31 wherein the anti-inflammatory compound comprises an antibody that results in the subject producing a reduced amount of TNF.

35. The method of claim 34 wherein the antibody is an anti-IL-17 antibody or an anti-IL-23 antibody.

36. The method of claim 34 wherein the antibody is an anti-TNF antibody.

37. A method of ameliorating immunosuppression associated with administering an anti-inflammatory compound to provide medical treatment, the method comprising:
administering the anti-inflammatory compound in an amount effective to provide the medical treatment; and
administering an IRM compound in an amount effective to ameliorate the immunosuppression associated with administering the anti-inflammatory compound.
